(19) United States
(12) Patent Application Publication
(10) Pub. No.: US 2003/0223942 A1
(43) Pub. Date: Dec. 4, 2003

(54) Aqueous composition containing high purity iron oxide

(75) Inventors: Rebecca Lister, St. Louis, MO (US); George Podolsky, Kirkwood, MO (US); Greg Furness, St. Louis, MO (US); Rick Gevemuehle, Arnold, MO (US); Frank Dunbar, O'Fallon, IL (US)

Correspondence Address: RHEOX, INC., WYCKOFFS MILL ROAD, P O BOX 700, HIGHTSTOWN, NJ, 08520

(73) Assignee: Elementis Pigments, Inc.

(21) Appl. No.: 10/156,637

(22) Filed: May 29, 2002

(51) Int. Cl. A61K 7/021; C09C 1/22; A23K 1/00

(52) U.S. Cl. 424/63; 426/635; 106/456

(57) ABSTRACT

An aqueous composition comprising water and high purity iron oxide preferably including a preservative is described. The composition is useful as a coloration ingredient in pharmaceuticals, cosmetics, foods, pet foods, and tobacco products which can be incorporated as a liquid conveniently into system of customers that desire a liquid high purity iron oxide ingredient or have been convinced to change from a dry high purity iron oxide ingredient to obtain the benefits of a liquid system.

### FIELD OF INVENTION

The present invention is directed to an aqueous composition containing high purity iron oxide pigments. The compositions involved are particularly useful for providing coloration and other properties to a large variety of pharmaceutical, food, pet food, cosmetics, personal care products and other systems where high purity, high quality products are necessary and where the customer desires a liquid product to incorporate into such customer's manufacturing processes.

### BACKGROUND OF INVENTION

In General

Since at least the middle of the Nineteenth Century to the very present day, iron oxide has been used as the pigment of choice in an ever expanding and increasing variety of systems. Natural iron oxide, actually was mined and used in paints before the American Civil War and such paint use continues, on a very large scale, into the Twenty First Century Waxes, coatings, inks, paper and a growing number of other new products continue to depend on iron oxide to provide the bright colors which some people believe define the vividness of modernity and our present time

Most recently iron oxide (and chromium oxide) have been used in the pigmentation of cosmetic and pharmaceutical products. Metal oxide pigments have been used in the form of a powder in these businesses until very recently when customer preferences for a liquid formulation became known

Powdered metal oxide pigments are dusty, thereby giving rise to health hazards and making storage and handling difficult. Also, the powders are not free flowing and so cannot readily be conveyed through pipes, which become blocked by the powder. Further, the poor flowing properties of powders makes it hard to meter them using for example auger screws to ensure the correct proportion of pigment to base material

An increasing number of iron oxide customers have simultaneously been expressing a preference for relatively new high purity iron oxides. This iron oxide product is manufactured to meet more modem rigid standards of reduced trace elements; many of which elements either have been identified with health problems or are of a type not applicable to uses where food or skin contact is involved.

Prior Developments

Similar powder problems are known in other industries (e.g., in the concrete industry). Such problems have been solved to a substantial extent by granulating iron oxide products into large size granules 100 s of times larger than iron oxide pigment particles Granules have been difficult to optimize and are the subject of an ongoing investigation by many companies with much prior art reflecting the search for a process that is both effective in producing useful granules and is relatively inexpensive to implement.

Most cosmetic, pharmaceutical, and food customers however have resisted granules and have expressed a preference for a liquid iron oxide delivery system which would provide ease of use in the customer's manufacture. Many systems such as cosmetic and pharmaceutical making are largely liquid and pumpable/pourable raw materials in a water base would be very welcome.

U.S. Pat. No. 5,401,313 issued to Elementis Pigments, Inc., the assignee of this invention, describes a spray drying process wherein a granule is created with an added step of coating the iron oxide pigment particles with electric charges through use of a coating is utilized. The granules of iron oxide is presently useful in coloring concrete.

U.S. Pat. No. 5,853,476 also issued to Elementis Pigments, Inc., shows a compaction process relying in a preferred embodiment on Bepex MS compactors to make iron oxide granules. While very effective, the process is relatively expensive. The patent teaches the use of recycling of oversize and undersize material streams in a process that both creates enhanced color saves the cost of waste disposal. The final product is a solid granule.

### OBJECT OF THE INVENTION

It is an object of the present invention to produce an aqueous dispersion of high solids content high purity iron oxide that is readily and very rapidly dispersible in a base medium, thereby eliminating dusting. It is a further important object to use in such dispersion from 5 to 80% high purity iron oxide.

It is a further object of the present invention to provide a process of manufacturing such dispersion and then selling such inventive products to customers as coloring ingredients in, for example, cosmetic, food, pet food, and pharmaceutical formulae

### DETAILED DESCRIPTION OF INVENTION

According to the present invention, there is provided an aqueous dispersion of high purity iron oxide pigment preferably containing one or more preservatives

The aqueous composition of this invention is a composition where water comprises from about 20% to about 95% by weight of the total composition—when we use the word aqueous, we mean a liquid system based on water. The water preferably used is itself of high purity and clarity. Mineral water including Evian and Evian-type water can be used in high end dispersions targeted to the cosmetic industry particularly lipsticks, face creams, rouges and mascaras. Water obtained from artesian wells or other sources not affected by urban pollution is also preferred Preferred for most food and pharmaceutical uses however would be tap water preferably subjected to at least one secondary impurity treatment.

The present invention relates to a iron oxide suspension with high solids content and to a process for the preparation thereof The present invention uses as the starting material high purity iron oxide pigment prepared for example through the reaction of iron salts with an oxygen-containing gas in a reactor utilizing well known processes for manufacturing iron oxide, slurried in water with or without the use of a dispersing agent.

The key to high purity lies generally in special selection of raw materials used to make a purified iron starting solution to make the iron oxide. Raw materials include specifically selected steel and acid. Steel selected for purity includes steel free of organic contaminants and low in heavy metals, for example, stampings from steel cans used for food products. Acids are selected from, for example, sulfuric and hydrochloric acid, that has not been regenerated from heavy metals containing processes.

Representative manufacturing processes to make high purity iron oxide can vary. Generally the processes may be categorized into two types 1) precipitation, and 2) thermal decomposition, such as calcination and gas phase chemical vapor deposition. Some products can be manufactured by a combination of these two general process routes.

1) Precipitation—General Description

Yellow, red, and black iron oxides are precipitated products that rely on careful control of a complex series of liquid-solid, gas-solid, and gas-liquid reactions. Nucleation and precipitation/crystallization kinetics are the preferred key to preparation of the correct chemical composition, particle size, particle size distribution, particle morphology, and ultimately, the desired color.

Both the yellow (goethite) and red (hematite) products are made from a modified version of the Penniman-Zoph process in which a nucleus or seed particle is grown to a target size The source of the nutrient for this particle growth is continuously provided by dissolution of iron. The iron is a specially-selected grade that is dissolved in an acidic ferrous sulfate solution and oxidized with finely-dispersed air. The primary distinction between the yellow and red processes is in the nature of the seed particle.

The following representative reactions (not balanced) depict the seed and growth (generation) stages of precipitation processes

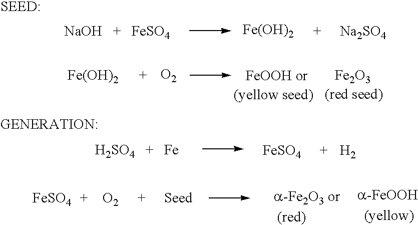

Following the seed generation stage, the yellow or red iron oxide slurry is filtered, washed, dried, milled, and packaged. An alternative yellow and red iron oxide process is a direct precipitation route. In such a process the nutrient is a preferred iron salt solution rather than the specially selected iron.

The black iron oxide process resembles the “Seed” phase of the reactions but must be conducted at a higher temperature and pH to precipitate magnetite:

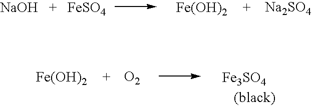

Terminal stage operations are similar to the yellow and red processes.

2) Calcination—General Description

Solid state reactions with strict control of gas-solid equilibria characterize the calcination manufacturing processes used to prepare the red and copperas red iron oxide product lines

Calcination of yellow iron oxide is a dehydroxylation of the oxyhydroxide to yield red iron oxide as depicted in the following reaction.

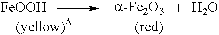

The copperas reds are prepared by a process that results in a hematite with the highest chemical purity and chroma.

A purified ferrous sulfate solution is further purified during evaporation and crystallization stages. The ferrous sulfate heptahydrate crystals (FeSO₄.7H₂O), also known as “copperas,” are dried and dehydrated to ferrous sulfate monohydrate (FeSO4.H₂O). The monohydrate is then oxidized during a calcination step to the hematite (α-Fe₂O₃) or copperas red iron oxide particle The by-product sulfur gases are recycled to the contact sulfuric acid plant. Further purification is achieved during subsequent washing steps followed by filtration, drying, milling and packaging.

The process can be summarized by the following chemical reaction (not balanced):

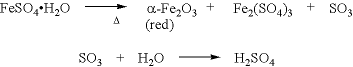

The inventive process comprises mixing (e g. blending, grinding and dispersing) such high purity iron oxide pigment made as described above, or by other known processes, with water to form an aqueous composition Useful for the high purity iron oxide pigments of the invention are a family of pigments made by the assignee of this invention and sold under the TruPure trademark, for example Tru Pure R2199 AP, a red iron oxide. Rockwood Specialties makes a yellow high purity iron oxide sold under the description Y50EC also useful for this invention. Bayer GmbH makes a yellow product (920Z) also useful BASF, a large German chemical company, makes a high purity black iron oxide useful for mascara called Sicovit Black 80E172.

Useful products include red, copperas red, black, green, blue-green, yellow, brown and blends therefore. These iron oxide pigments have the following technical specification or characteristics. It is to be understood that such individual pigment particles (because of their very small size) can “clump” together to form larger pigment agglomerates

1. Average particle size—from 0.01 to 1.30 μm

2. Specific surface area—from 3.0 to 200 m²/gram

3 Specific gravity—from 3 20 to 5.20

In order to be useful in the dispersions of this invention particularly for cosmetic, pharmaceutical, food including pet food, tobacco and personal care customers, these products must critically contain pigment with substantially lower maximum levels of impurity levels than normal as follows:

1. Not to exceed 20 ppm lead preferably not to exceed 10 ppm

2. Not to exceed 5 ppm arsenic preferably not to exceed 3 ppm

3. Not to exceed 3 ppm mercury preferably not to exceed 1 ppm

Useful high purity iron oxide particles are formed using known techniques such as previously described, such that the resultant pigments have acceptable, i e., reduced, levels of trace metals such as arsenic, mercury and lead. These levels are substantially lower than those found in iron oxide sold for use in the paint and coatings industry for example, where toxicity is not a primary concern. Pigments, having the requisite levels of these trace metals for use in the present invention, are therefore referred to in the present specification as substantially “pure” or as “high purity” This substantially “pure” material is suitable for application to human skin since the trace metal content is maintained at or below the levels set forth above, that is, below levels which are very, very unlikely to cause dangerous effects in humans or animals.

Dispersants and other chemicals may be added to the inventive aqueous compositions including grinding aids such as glycerine, preservatives such as potassium sorbates, citric acid and combinations thereof to produce sorbic acid, anti-settling additives, wetting agents such as lecithin, flavor ingredients, other pigments such as zinc oxide and titanium dioxide, and rheological additives just for example. Preservative—containing dispersions are preferred The dispersions should as a rule not contain appreciable amounts of organic solvents such as propylene glycol, xylene, toluene, or herbicides and biocides.

In a preferred embodiment, applicants' substantially pure iron oxide particles are formed having a substantially spherical shape. A variety of other shapes, such as acicular, oval and rhomboids have also been found to provide acceptable cosmetic use, however, any shape may be utilized in the formulations of the invention as well, although as noted above, spherical particles are the most preferred.

The “purity” of these iron oxide pigments renders formulations containing this material suitable for application to human skin without danger due to transdermal absorption of trace metals. It also permits use in foods of an ingredient of great safety and effectiveness in providing color to such formulation In addition the invention provides an aqueous vehicle of great pumpability and flowability to permit a customer flexibility in manufacture without the safety or environmental risks involved in the handling of powder.

The substantially pure iron oxide particles are incorporated into a aqueous dispersion preferably with one or more preservatives Such dispersions may then be incorporated by known mixing methods into a variety of cosmetic products such as lipstick, eyeshadow, foundations, moisturizers, rouges and the like to form cosmetics having an increased acceptance to discerning health-oriented customers. They are also particularly useful for food and pet food companies who want to impart coloring to their products to provide, for example, a “meaty look” for dog and cat food without any likelihood of harm to pets.

Iron oxide particles of the size and morphology described herein, with such reduced levels of trace metal contamination, have been previously known in the art Dispersions of low purity iron oxide pigments in water have also been known since at least the early 1900's and “relatively” high purity type iron oxides has been available on the market since the 1930's. It is speculated that persons in the art believed that adding such iron oxides to water would result in an unpure dispersion because of the generation of harmful acids It is also believed that chemists working in the field thought that undesirable side reactions of the iron oxide would occur in water decreasing their purity and that a liquid dispersion in water would promote the formation of mold, fungus, or microbial activity.

There is no teaching or suggestion that applicants are aware of to utilize iron oxide particles of the type described herein in an aqueous dispersion for applications such as those contemplated by applicants, namely as a component of: 1) cosmetic formulations, 2) food compositions, 3) pharmaceuticals and 4) pet foods particularly foods made for cats, dogs and other domestic animals capable of providing an enhanced degree of satisfaction. Thus the use of applicants' pure iron oxide dispersion in the manner indicated provides unexpected results with regard to the ability of this material to afford the customer both ease of handling and satisfaction of having met all environmental concerns

### EXAMPLE I

In this Example, the percentages stated are by weight based on the weight of the pigment used. The following steps are used to illustrate the invention herein.

Step 1—Mixing

Components:

1. 5-80% by weight (preferably 20-60%) high purity iron oxide pigment (red, yellow, black, brown or blends).

2. 20-95% by weight of water.

3. 0-5% by weight (preferably 3%) of one or more preservatives

4. 0-15% by weight (preferably 8%) of one or more dispersants.

5. 0-15% by weight (preferably 8%) ofone or more anti-settling additive.

Iron oxide was loaded into a media mill where liquid mixture of water and other ingredients were added. Mixing preferably occurs using a high-speed disperser or media mill; time of mixing preferably 5 minutes to 30 minutes; 100 to 3000 revolutions per minute if a high speed disperser is used

Discussion of Results:

The result is a high purity iron oxide dispersion that meets FDA specifications for food, pet food, cosmetics, pharmia, and personal care. It will also meet specifications for European directive E-172. The improvements include:

Allows for batch to batch uniformity.

Better color work up with less pigment 10-30% less pigment

No dusting.

No cross contamination of equipment or raw materials.

Less labor intensive

hookup pump

no clean up

economical process

### EXAMPLE II

The following example shows the manufacture of a aqueous dispersion according to this invention designed to be particularly useful for pet foods.

Red Pigment Dispersion for Pet Food

| Raw Materials | grams | % | Observations |
|---|---|---|---|
| water | 75 | 21.60 | shear thins |
| glycerine 99.7% | 12 | 3.50 | no syneresis |
| Turn on disperser | | | no settling |
| Thermolec WFC | 25 | 7.20 | |
| High speed grind for 10 | | | |
| minutes 500 rpms | | | |
| High Purity Red Iron Oxide | 225 | 64.90 | |
| Slowly add pigment-This may | | | |
| take up to 60 minutes | | | |
| Grind for 30 minutes (1500-2000 rpms) | | | |
| potassium sorbate preservative | 2.8 | 0.80 | |
| 25% wt. Soln. | | | |
| Let down, grind 10 minutes | | | |
| citric acid preservative 25% wt. Soln. | 6.8 | 2.00 | |
| (add drop-wise) | | | |
| Grind for 10 minutes (1500-2000 rpms) | | | |
| | 346.6 | 100.00 | |

Physical Properties
Solids: 76.63%
Hegman: 6.0
Density: 17.15 lbs/gal.
pH: 4.37
Viscosity: Brookfield, #4 spdl, 60 rpms 3200 cP
Hercules pass, 22, narrow cycle

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof

What is claimed is:

1. An aqueous composition useful for providing coloration comprising:
a) from 20 to 95% by weight of water; and
b) from 5 to 80% by weight of iron oxide pigment, said pigment having an average particle diameter of from about 0.01 to 1.30 μm and which pigment contains

| | |
|---|---|
| lead | not to exceed 20 ppm; |
| arsenic | not to exceed 5 ppm; and |
| mercury | not to exceed 3 ppm; |

said iron oxide pigment being substantially dispersed in said aqueous composition.

2. The composition of claim 1 wherein said iron oxide pigment comprise less than about 60% by weight of said composition

3. The composition of claim 1 wherein said iron oxide pigment comprise less than about 25% by weight of said composition.

4. The composition of claim 1 wherein one or more chemical compositions selected from the group consisting of preservatives, dispersants, anti-settling agents, flavor ingredients and rheological additives have been added to the composition.

5. The composition of claim 4 wherein the chemical composition is one or more preservatives.

6. The composition of claim 1 wherein the iron oxide pigment has the following characteristics
1. average particle size—from 0 01 to 1 30 μm,
2 specific surface area—from 3.0 to 200 m²/gram, and
3. specific gravity—from 3 20 to 5 20.

7. An aqueous composition useful for providing coloration comprising:
a) from 20 to 95% by weight of water;
b) from 5 to 80% by weight of iron oxide pigment, said pigment having an average particle diameter of from about 0.01 to 1.30 μm and which pigment contains:

| | |
|---|---|
| lead | not to exceed 20 ppm; |
| arsenic | not to exceed 5 ppm; |
| mercury | not to exceed 3 ppm; and |

c) one or more preservatives.

8. The composition of claim 7 wherein one or more of the preservatives are selected from the group consisting of potassium sorbates, citric acid, sorbic acid or mixtures there.

9. A formulation containing the aqueous composition of claim 1.

10. The formulation of claim 9 selected from the group consisting of cosmetic formulations, pharmaceuticals, foods including pet foods and tobacco products.

11. A method of making an aqueous composition useful for providing coloration comprising
a) preparing iron oxide pigment, said pigment having an average particle diameter of from about 0 01 to 1.30 μm, which pigment contain:

| | |
|---|---|
| lead | not to exceed 10 ppm; |
| arsenic | not to exceed 5 ppm; and |
| mercury | not to exceed 3 ppm; and |

b) and mixing such iron oxide pigment into water in an amount of from 20 to 95%, thereby forming the aqueous composition.

12. The method of claim 11 wherein the iron oxide pigment contain

| | |
|---|---|
| lead | not to exceed 10 ppm, |
| arsenic | not to exceed 3 ppm; and |
| mercury | not to exceed 1 ppm. |
